(19) United States
(12) Patent Application Publication
(10) Pub. No.: US 2009/0264898 A1
(43) Pub. Date: Oct. 22, 2009

(54) Steerable Endovascular Retrieval Device

(75) Inventors: Sean Miller, Danvers, MA (US); Paul Squadrito, Rowley, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/104,671

(22) Filed: Apr. 17, 2008

(51) Int. Cl. A61B 17/50; A61B 17/00

(52) U.S. Cl. 606/130

(57) ABSTRACT

A retrieval catheter for retrieving endovascular devices from the lumen of a blood vessel includes a controllable steerable distal tip.

### FIELD OF THE INVENTION

The invention relates to retrieval catheters for retrieving endovascular devices from the lumen of a blood vessel.

### BACKGROUND OF THE INVENTION

A number of intravascular procedures, such as angioplasty, atherectomy and stenting, among others, include the placement of a distal protection filter within the blood vessel at a location distally of the intended treatment site. The distal filter is intended to entrap embolic debris that might become loosened as a result of the procedure. The filter prevents such debris from flowing downstream where it may embolize and obstruct blood flow, potentially causing significant damage or death. Although distal protection devices may take a variety of configurations, a typical filter includes an umbrella or parachute-like device that is navigated to a location in the blood vessel distally beyond the targeted treatment site and then is opened to engage the vascular wall and span the vascular lumen. The device includes a mesh or other filter element adapted to entrap embolic material while enabling blood to flow. Commonly, such a filter is mounted on the distal end of a guidewire that also can function during the intravascular procedure to guide various catheters and other devices to and from the target site while maintaining the filter downstream of the target site.

After the intravascular procedure has been concluded, the filter and any embolic debris that it may have captured must be removed from the patient. Typically that involves the use of a retrieval catheter that is essentially tubular at its distal end and has an open distal port adapted to be advanced toward the filter to engage, progressively, proximal portions of the filter to draw the peripheral portions of the filter together to cause the filter to collapse toward the guidewire. The collapsed filter, containing the entrapped debris is drawn through the distal port into the distal portion of the retrieval catheter to the extent possible depending upon the volume of debris contained within the filter. The retrieval catheter, with the retrieved filter and its associated guidewire then can be removed from the patient.

The distal port of the retrieval catheter must be large enough to engage the proximal portions of the filter to collapse the filter as the catheter is advanced and then to receive the collapsed filter in the distal end of the catheter. Consequently, the distal end of the catheter has an open mouth and presents a risk of becoming caught or snagged on irregularities in the vasculature as the catheter is advanced to the target site. If, as is commonly the case, one or more stents has been placed in the blood vessel there is a risk of the leading edge of the catheter catching on edges or projections of the stent. Additionally, difficulties may be encountered during the navigation of the catheter toward the target site, should the catheter engage bifurcations or other tortuous portions of the vascular anatomy en route.

Among the proposals to deal with the foregoing risks is that described in PCT Application Publication No. 2006/065516 (Callol) in which a retrieval catheter is provided with a tapered dilator tip at its distal end. The tapered tip projects beyond the distal end of the catheter to create a transition from the distal mouth of the catheter to reduce the risk of the catheter tip becoming caught on vasculature or an intraluminal device, such as a stent. A retraction wire is attached to the dilator tip to enable the tip to be pulled back into the tubular catheter shaft sufficiently to make room to capture and contain the filter. One embodiment discloses a tip deflection wire that can be tensioned to cause the tip to be bent in one direction in a plane to reorient the tip.

It would be desirable to provide a retrieval catheter having a retractable leading tip with the ability to be manipulated by the clinician selectively and omni-directionally to provide additional control to avoid obstructions in the vasculature as well as to advance the distal end of the device through a treated target region.

### SUMMARY OF THE INVENTION

A retrieval catheter in accordance with the invention includes a distal tube adapted to engage the proximal portions of a vascular filter to gather the margins of and collapse the filter and then to receive the filter in the distal portion of the catheter. The catheter includes a steerable tip that is contained within and partly projects out of the distal end of the lumen of the distal tube and may be withdrawn proximally into the lumen by a control rod that is attached to the steerable tip. The rod extends to the proximal end of the catheter where it is attached to a rotatable control knob. The steerable tip may have a curved distal segment and can be rotated by rotating the control rod and knob at the proximal end. Thus, as the catheter advances toward a bifurcated or sharply tortuous portion of the vasculature, as determined by imaging, the distal tip can be controllably steered to direct the catheter in the intended direction to avoid an obstruction. Similarly, when the device is advanced through the target site toward the device to be retrieved, the tip can be rotated in either direction to avoid interference with potential obstructions such as portions of a stent.

### DESCRIPTION OF THE DRAWINGS

The invention will be appreciated more fully from the following further description, with reference to the accompanying drawings in which:

FIG. 1 is a somewhat diagrammatic longitudinal sectional illustration of a retrieval catheter in accordance with the invention;

FIGS. 1A and 1B illustrate a longitudinal section of the side port region of the catheter showing one arrangement for guiding the proximal end of a guidewire out of the side port;

FIG. 1C is an illustration similar to FIG. 1A illustrating an alternate configuration for the side port;

FIG. 2 is an enlarged fragmented illustration of the proximal and distal ends of the catheter of FIG. 1 with the distal tip having been rotated to an orientation different from that of FIG. 1;

FIG. 3 is an illustration of the distal end of the catheter as seen along the line 3-3 of FIG. 2.

FIG. 4 is a sectional illustration of the distal end of the catheter as seen along the line 4-4 of FIG. 3;

FIG. 5 is a diagrammatic cross-sectional illustration of the distal end of the catheter showing the distal tip withdrawn into the distal end of the catheter;

FIGS. 6-9 illustrate the sequential operation of the device in retrieving a distally placed embolic filter;

FIG. 10 is a sectional illustration of the distal portion of an alternate embodiment of a rapid exchange shaft that may be used to practice the invention;

FIG. 11 is a diagrammatic illustration of an alternative embodiment of the steerable tip;

FIGS. 11A and 11B are end views of the top of FIG. 11 as seen along the line 11A, B-11A, B of FIG. 11;

FIG. 12 is a diagrammatic side elevation of another embodiment of the steerable tip; and

FIG. 12A is a diagrammatic illustration of the tip of FIG. 12 having been manually bent to a selected shape.

### DESCRIPTION OF THE ILLUSTRATIVE EMBODIMENTS

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

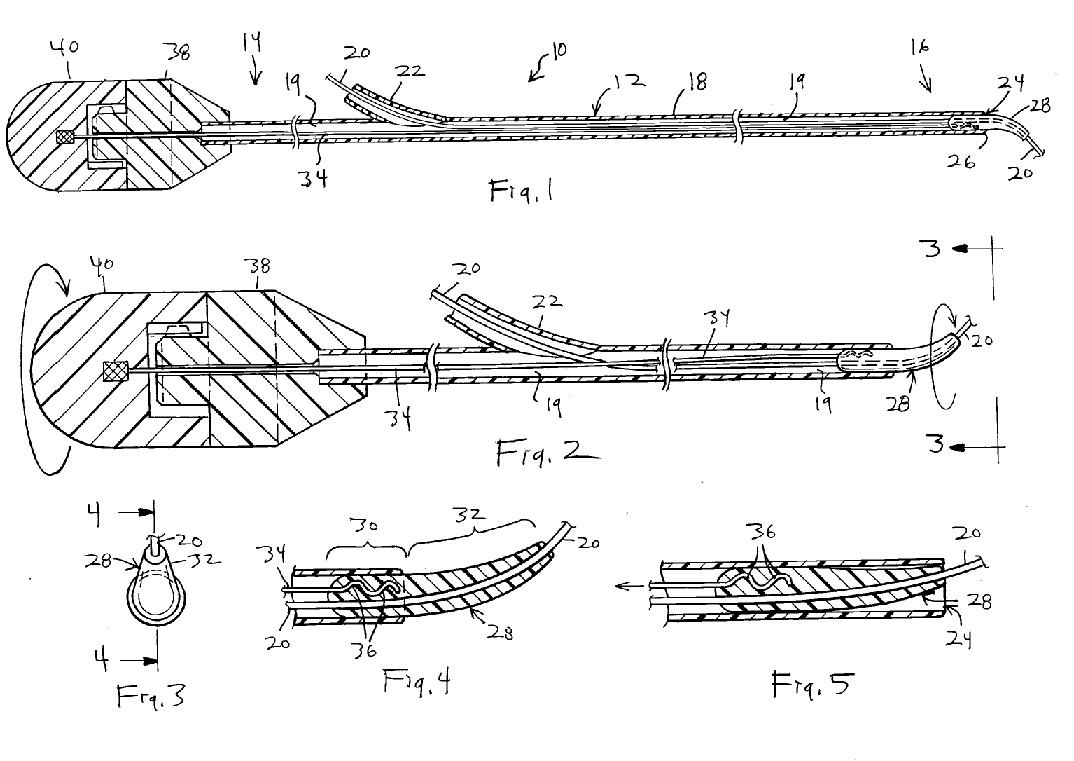

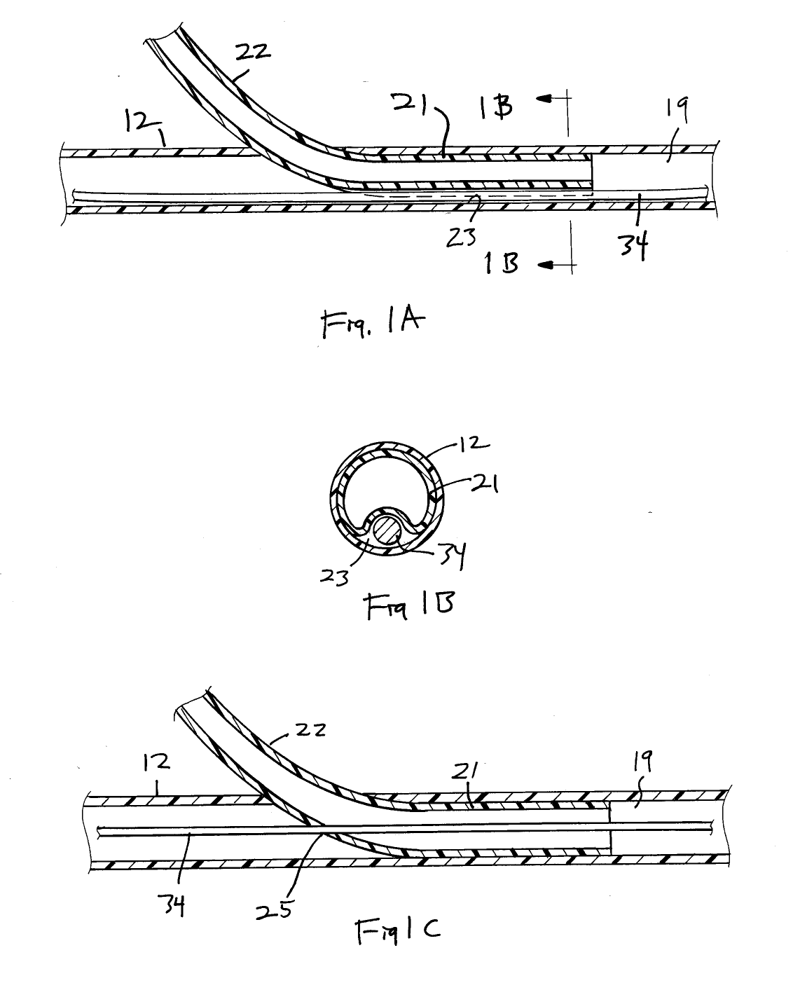

FIG. 1 illustrates an embodiment of the invention as it may be incorporated into a rapid exchange or “single operator” catheter. The catheter 10 includes an elongate tubular shaft 12 having a proximal portion 14 and a distal portion 16. The shaft 12 may incorporate any number of constructions known to those skilled in the art, such as the single lumen tube 18. The tube 18 may be formed from a polymeric material having sufficient flexibility to track over a guidewire 20 and be advanced to the target site. In the rapid exchange embodiment shown in FIG. 1, the single lumen tube 18 is formed to include a proximal guidewire port 22 located distally of the proximal end of the catheter. The proximal guidewire port may be formed in a variety of configurations. FIG. 1A illustrates the port as defined by a short tubular member 21 having a distal end disposed within the lumen 19 and having a proximal portion 22 that protrudes outwardly through the side of shaft 12. As shown in FIG. 1B, the intraluminal portion of the tube 21 may be formed with a longitudinal channel disposed against the inner surface of shaft 12 to form a passageway 23 adapted to receive and enable longitudinal and rotational movement of a control rod 34 described below. FIG. 1C illustrates an alternate configuration in which the control rod 34 is passed through the distal portion of the tube 21 and then through an aperture 25 formed in the tube 21. The segment of lumen 19 within distal portion 16 defines a distal lumen, which terminates in a distal opening 24. The distal opening 24 is adapted to define a port through which the distal filter can be collapsed and retrieved, as described below. The distal edge 26 of the shaft that defines the port 24 preferably is radiused or otherwise shaped to reduce the risk of becoming snagged or caught as it is advanced through the vasculature.

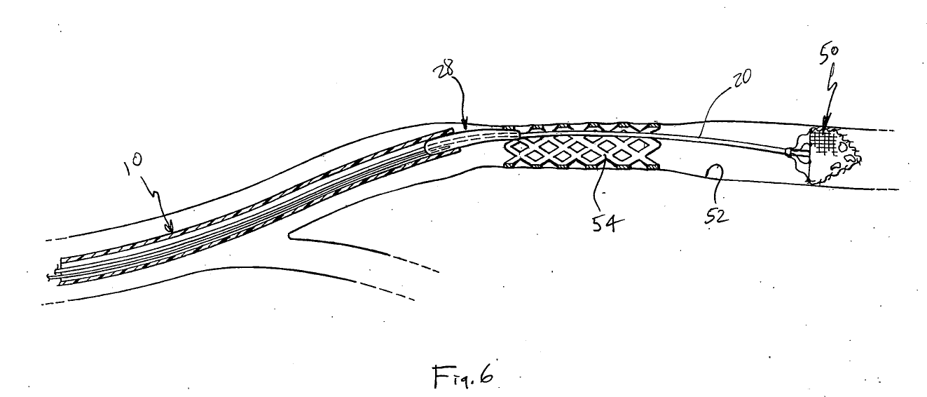

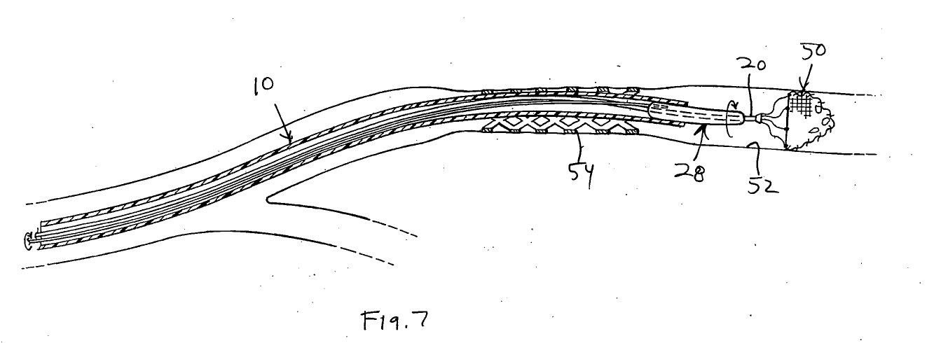

The catheter also includes, at its distal end, a steerable tip 28, one embodiment of which is illustrated in FIGS. 1-5. The steerable tip includes a lumen 29 adapted to receive the guidewire 20. Tip 28 may be formed to include proximal and distal segments 30, 32, the proximal segment 30 being cylindrical and having an outer diameter sized for a close, moveable fit within the distal portion of the cylindrical catheter lumen 19. Distal segment 32 is tapered and, in this embodiment, is pre-formed to include a bend. In one position, distal segment 32 protrudes from distal port 24 to function as a leader providing a gradual increase in diameter at the distal end of catheter 10 from guidewire 20 to the small step at distal edge 26. The tip 28 is rotatable and translatable within the distal portion of the lumen 19. The rotational and longitudinal position and orientation of the steerable tip 28 is controlled by an elongate control rod 34, the distal end of which is embedded in the steerable tip 28, e.g., in proximal segment 30. The embedded distal end of the control rod may be formed with irregularities 36 to provide a secure connection to assure that when the control rod 34 is rotated, the distal tip 28 will rotate to the same degree. The control rod is constructed to have flexibility and torsional stiffness to enable the rod 34 to advance to the target site as well as to transmit to the steerable tip 28 substantially all of the rotation applied at the proximal end of the control rod 34. The proximal end of the catheter may include a fitting 38 secured to the proximal end of the shaft 12. A control knob 40 is rotatably disposed with respect to the fitting 38 and is secured to the proximal end of the control rod 34. Rotation of the control knob 40 in either direction thus rotates the rod 34 and, in turn, the distal tip 28 in that direction to orient the distal segment 32 of the tip 28 omni-directionally. The longitudinal position of the steerable tip 28 also is controllable by the control rod 34 and knob 40. The knob 40 is movable longitudinally with respect to the proximal fitting 38. Optionally, a limiting feature to limit the extent to which the control knob can be retracted may be employed.

The steerable tip 28 should be flexible sufficiently to reduce the risk of trauma even if it contacts tissue. It may be insert molded over the distal end of the control rod. The tip may be formed from low density polyethylene or other appropriate flexible low friction polymer that will facilitate rotation of the proximal segment of the tip within the distal end of the tube. The tip may be filled with a radiopaque material to aid fluoroscopic visualization of the location and orientation of the tip. The tip also should be sufficiently flexible so that it will deform and straighten when withdrawn into the distal end of the sheath. Alternately, the tip and distal end of the catheter tube may have relative flexibilities such that the tube is deformed by the distal segment 32 when the tip 28 is retracted into the tube.

Although the above-described embodiment illustrates a catheter shaft formed from a polymeric single-lumen tube 18 that extends from the proximal to the distal end of the catheter, those familiar with the art will recognize that other rapid exchange configurations may be employed as well. It also should be understood that the principles of the invention may be incorporated into an over-the-wire catheter in which the guidewire lumen extends fully through and out of the proximal end of the catheter.

FIG. 10 illustrates, in fragmented sectional form, a rapid exchange catheter in which the majority of the length of the catheter is formed by a hypotube 42 having a distal end to which a more flexible polymeric tube 44 is attached. The hypotube 42 may be covered with a polymeric jacket, for example, polyethylene block amide copolymer (PEBA). The juncture 45 of the distal portion of the hypotube 42 and the proximal portion of the distal tube 44 may be made according to a variety of techniques known to those familiar with the art. For example, the proximal end of the distal tube 44 may include a sleeve 47 that defines a proximal guidewire port 22′. The juncture 45 may be secured by a suitable adhesive and also may be secured with a heat shrink tubular film that surrounds the proximal end of the distal tube 44, the sleeve 47 and the distal end of the hypotube 42. The distal end of the hypotube preferably is skived, viz., cut at a shallow oblique angle to provide a smooth transition in flexibility at the juncture region 45. The steerable tip 28 may be mounted at the distal end of the distal tube 44 in the same manner as described above in connection with the embodiment of FIGS. 1-5. The control rod 34 extends proximally through the lumen within the hypotube 42 and is connected to the control knob 40 as described above.

The proximal end of the distal tube 44 is formed to include a proximal guidewire port 22′. The tube 44 contains a portion of the guidewire 20 and the control rod 34. The distance between the proximal port 22, 22′ and the distal opening 24 may be selected as desired, depending on the particular portion of the anatomy into which the device is to be used. The length should be selected to accommodate the possibility of increasingly tortuous vascular anatomy in the more distal regions of the vasculature.

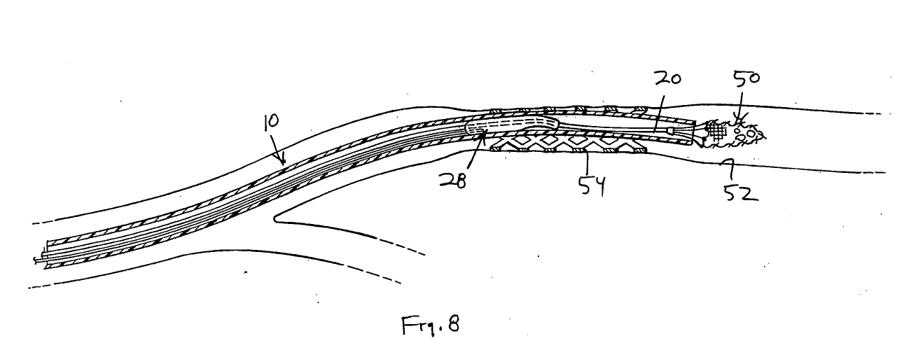

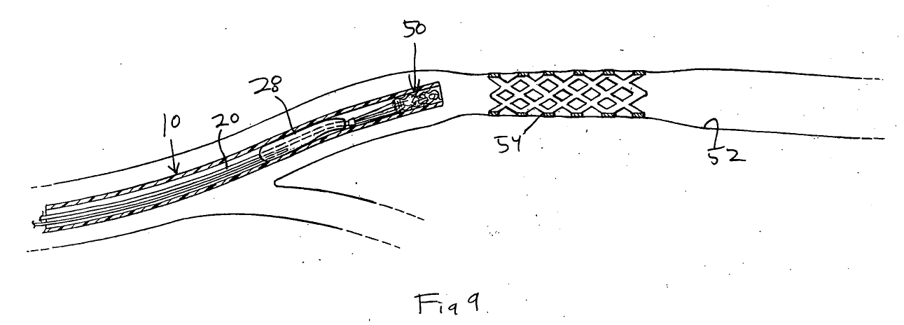

FIGS. 6-9 illustrate the manner in which the device is used to retrieve a distal embolic filter 50. The filter shown is of the type mounted to the distal end of a guidewire 20 and will have been placed within the lumen 52 of the blood vessel before or during the intravascular procedure. FIGS. 6-9 illustrate the use of the device in which an endovascular stent 54 has been placed in the lumen 52 by a catheter (not shown) slidable over filter guidewire 20. After the stent has been delivered and deployed, the stent delivery catheter will be withdrawn over the guidewire 20. With the distal segment 32 of steerable tip 28 protruding from distal port 24, the filter retrieval catheter 10 then is advanced by back loading the steerable tip 28 onto the proximal end of the guidewire 20 and advancing the catheter 10 along the guidewire 20. The proximal end of the guidewire 20 may be guided into the side port 20 by a ramp, defined by a tube. The tubular ramp may, in one embodiment, include an aperture through which the control rod may pass or may be formed to include a channel for the control rod. As the retrieval catheter 10 is advanced through the vasculature, the orientation of the steerable tip 28 may be controlled from the proximal fitting to avoid the distal portion of the catheter catching on an intraluminal surface or on part of the stent 54. The procedure is conducted under imaging, such as fluoroscopy, with contrast agent being supplied through the guide catheter (not shown) through which the procedural catheters and the retrieval catheter are guided into the vasculature. As the steerable tip 28 approaches the stent 54, the orientation of the tip may be controlled by rotating the control rod. As shown in FIG. 7, the retrieval catheter is passed through the stent as the tip 28 has been advanced toward the filter. As shown in FIG. 8, the control rod 34 is withdrawn proximally, causing the distal tip 28 to be retracted into the distal lumen of the retrieval catheter. The tip is withdrawn sufficiently to leave a distal portion of the distal lumen of the retrieval catheter open to receive the distal filter. In that configuration, the retrieval catheter is advanced while the longitudinal position of the guidewire 20 is held fixed. As shown in FIG. 8, it may be seen that the distal edge of the catheter has advanced over the proximal portion of the filter, causing the peripheral portions of the filter to be drawn inwardly so that continued relative movement of the filter and distal end of the catheter causes the filter to be drawn into the distal end of the catheter lumen. FIG. 9 illustrates the filter having been collapsed and drawn into the retrieval catheter with the retrieval catheter in the process of withdrawal from the patient.

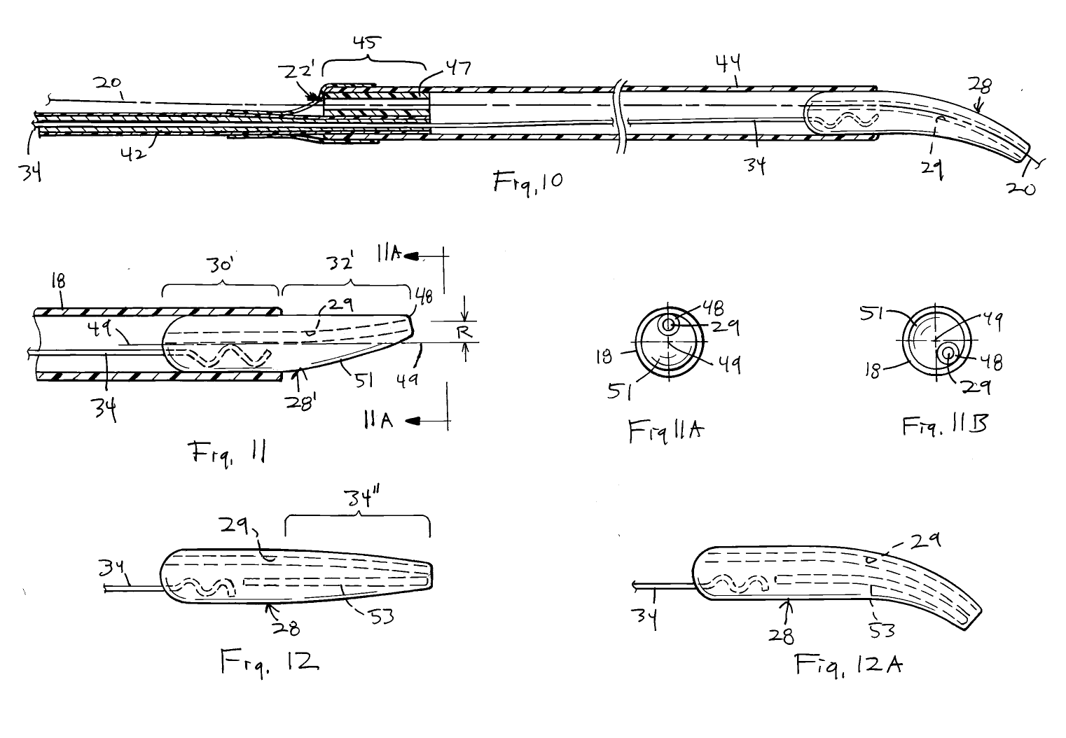

Other configurations may be employed for the tip. FIG. 11 illustrates an embodiment in which the steerable tip 28′ has a distal segment 34′ that remains within an imaginary extension of the cylindrical dimension defined by the lumen at the distal tube. The distal segment 32′ of the steerable tip defines an asymmetric taper in which the distal end 48 of the tip is spaced radially from the central longitudinal axis 49 of the proximal portion 30′ of the tip 28′. In this embodiment, the distal opening at the end of the tip may be located a spaced radial distance R from the center line 49. With this embodiment, the asymmetric taper of the tip defines a compound, curved surface 51, the orientation of which can be varied in response to rotation of the tip. FIG. 11B illustrates this embodiment of the tip in a partially rotated orientation as compared to FIG. 11A.

FIG. 12 illustrates another embodiment of the tip in which the tip includes a formable distal segment 34″. The distal segment is formable manually by means of a malleable, formable ribbon or insert 53 within the tip. The insert 53 may be formed from stainless steel, platinum or other suitable ductile, radiopaque material. With this arrangement the clinician can determine fluoroscopically, the degree of orientation of the desired shape for the distal segment of the tip and may form, manually, the tip accordingly.

It should be understood that the foregoing description of the invention is intended merely to be illustrative thereof and that other embodiments, modifications and equivalents may be apparent to those skilled in the art while remaining within the scope of the invention.

We claim:

1. A steerable endovascular retrieval device comprising:
an elongate tubular member having proximal and distal ends and a distal lumen terminating in an opening at the distal end;
a steerable tip mounted in the distal lumen, the tip including proximal and distal segments, the proximal segment being cylindrical and having a longitudinal axis, the tip being rotatable about the axis and being slidable within the distal lumen, the distal segment of the tip being extendable from the distal opening of the tubular member and being tapered and terminating in a distal end that is radially spaced from the longitudinal axis of the proximal segment of the tip whereby rotation of the tip within the distal lumen enables selective orientation of the distal end of the tip;
a guidewire lumen extending through the steerable tip and terminating in an opening at the distal end of the tip;
a control wire secured to the steerable tip and extending proximally therefrom and beyond the proximal end of the tubular member to enable the steerable tip to be selectively rotated and retracted into the distal lumen, the control wire having sufficient flexibility and torsional stiffness to transmit, controllably, to the steerable tip substantially all rotation applied at the proximal end of the control rod.

2. The device as defined in claim 1 wherein the radial spacing of the tip of the distal segment does not extend beyond an imaginary envelope defined by a cylindrical extension of the proximal segment of the steerable tip.

3. The device as defined in claim 2 wherein the distal segment of the steerable tip has an asymmetrical taper.

4. The device as defined in claim 1 wherein the radial spacing extends radially beyond an imaginary envelope defined by a cylindrical extension of the proximal segment of the steerable tip.

5. The device as defined in claim 1 wherein the distal segment of the steerable tip has a preformed curve.

6. The device as defined in claim 5 wherein the flexibility of the pre-curved distal portion enables it to flex to a more straightened configuration when it is withdrawn into the distal end of the tubular member.

7. The device as defined in claim 6 wherein the distal end of the tubular member has a flexibility such that it flexes in response to withdrawal of the steerable tip into the lumen of the tubular member.

8. The device as defined in claim 1 wherein the distal segment of the steerable tip has an embedded manually formable member whereby the tip may be manually shaped to a selected contour that is maintained by the manually formable member.

9. The device as defined in claim 1 further comprising a fitting at the proximal end of the tubular member and a rotatable member disposed on the fitting and connected to the proximal end of the control member, the handle being rotatable and translatable with respect to the fitting.

10. The device as defined in claim 9 further comprising means for limiting the extent of rotation of the knob with respect to the fitting thereby limiting the degree to which the control rod and steerable tip can be rotated.

11. The device as defined in claim 1 wherein the proximal segment of the steerable tip substantially fills a diameter of the distal lumen.
